(19) United States
(12) Patent Application Publication
(10) Pub. No.: US 2005/0143774 A1
(43) Pub. Date: Jun. 30, 2005

(54) Laparoscopic needle manipulator

(76) Inventor: Oscar Rafael Polo, Portland, OR (US)

Correspondence Address: Joseph Pugh, 3012 SW Dickinson Street, Portland, OR, 97219, US

(21) Appl. No.: 10/972,221

(22) Filed: Oct. 21, 2004

Related U.S. Application Data
(60) Provisional application No. 60/513,297, filed on Oct. 21, 2003.

(51) Int. Cl. A61B 17/04

(52) U.S. Cl. 606/205

(57) ABSTRACT

A laparoscopic needle manipulator includes a shaft, a handle at a first end of the shaft, and a grasper to hold an object at a second end of the shaft, where the handle controls opening and closing of the grasper.

### CROSS REFERENCE TO RELATED APPLICATION

This application claims the benefit U.S. Provisional Patent Application No. 60/513,297 filed on Oct. 21, 2003.

### TECHNICAL FIELD OF THE INVENTION

Embodiments of the invention are generally related to the field of medicine, and, in particular, to laparoscopic surgery.

### BACKGROUND OF THE INVENTION

When performing laparoscopic (i.e., minimally invasive) suturing, a curvilinear needle (also referred to herein simply as a needle) is typically used. During laparoscopic suturing, a needle driver is typically used to insert the needle into tissue, while a tissue grasper is used to receive the needle as it passes through the tissue, grasp the tissue into which the needle is being inserted, and help tie the knot in suture material (such as thread) when suturing is complete. In addition, a surgeon frequently encounters a needle that is not at a proper angle and/or orientation relative to the tissue through which the needle is to be driven. In that case, the tissue grasper is used to orient the needle so that it is in the correct position for the needle driver to insert it into the tissue.

A disadvantage of a tissue grasper is that in order to change the orientation of a curvilinear needle, the needle must be flipped over and then turned right side up. For example, this may involve rotating the needle on its Z-axis and then on its X-axis. Once properly oriented, the needle driver is used to take the needle from the jaws of the tissue grasper and drive the needle through tissue.

Changing the directional orientation of the needle, as well as using the needle driver to take the needle from the jaws of the tissue grasper, is often difficult to accomplish without having the needle fall from the tissue grasper. A needle that falls from the tissue grasper typically lands on top of or between the folds of an organ, for example, the small bowel. Using a tissue grasper to retrieve a fallen needle is often challenging because of the difficulty in retrieving the needle without grasping some of the tissue and/or injuring an organ while picking up the needle.

Another disadvantage of using the tissue grasper is that it cannot cut suturing material. Consequently, a cutting instrument is needed. The cutting instrument may be used while the ends of other instruments are still in the body, in which case another access point is made to accommodate the end of the cutting instrument, in addition to the access points for the needle driver and the tissue grabber. Alternatively, the cutting instrument may replace the needle driver or the tissue grasper, in which case the end of one of those instruments is removed from the body and replaced with the end of the cutting instrument. Among other things, the disadvantages of the instruments described above increase the amount of time and the risk of injury involved in performing laparoscopic surgery.

### BRIEF DESCRIPTION OF THE DRAWINGS

Embodiments of the invention are illustrated by way of example, and not by way of limitation, in the figures of the accompanying drawing.

FIG. 1 illustrates one embodiment of a laparoscopic needle manipulator.

### DETAILED DESCRIPTION

A laparoscopic needle manipulator is described. In the following description, for purposes of explanation, numerous specific details are set forth. It will be apparent, however, to one skilled in the art that embodiments of the invention can be practiced without these specific details. In other instances, structures and devices are shown in block diagram form in order to avoid obscuring the understanding of this description.

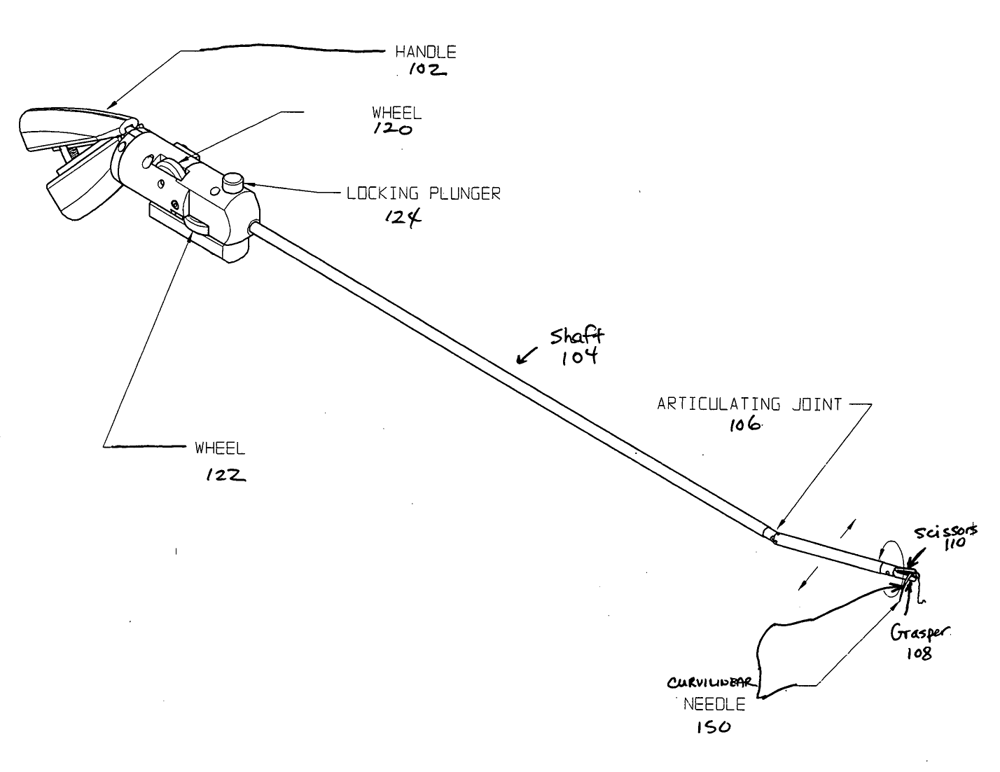

FIG. 1 illustrates one embodiment of a laparoscopic needle manipulator. Needle manipulator 100 includes handle 102, shaft 104, articulating joint 106, needle grasper 108 and scissors 110. Handle 102 may be coupled with or integrated as part of one end of shaft 104. Handle 102 may be a permanent part of shaft 104, or may be removable from shaft 104 in whole or in part. Grasper 108 and scissors 110 may be coupled with or integrated as part of another end of shaft 104. Grasper 108 and scissors 110 may be a permanent part of shaft 104, or may be removable from shaft 104 in whole or in part.

Handle 102 controls the opening and closing of grasper 108 and scissors 110. Articulating joint 106 enables shaft 104 to bend in any number of directions perpendicular to the axis of shaft 104. For this purpose, one embodiment of needle manipulator 100 includes wheel 122, which causes articulating joint 106 to bend, thereby enabling shaft 104 to bend in any number of directions perpendicular to shaft 104.

In addition, an end of shaft 104 may rotate in whole or in part. For this purpose, one embodiment of needle manipulator 100 further includes wheel 120, which when rotated causes grasper 108 and scissors 110 to rotate. Further, needle manipulator 100 includes locking plunger 124, which causes wheel 120 and/or wheel 122 to be held in a particular position so that they cannot be rotated.

Grasper 108 may be used, among other things, to receive curvilinear needle 150 as it passes through tissue, set curvilinear needle 150 so that a needle driver can grasp curvilinear needle 150 in a proper orientation for inserting it into tissue, grasp the tissue through which curvilinear needle 150 is being driven, and tie a knot when laparoscopic suturing is complete. Among other things, scissors 110 may be used cut suturing material.

Grasper 108 and/or scissors 110 are magnetized. Because grasper 108 and/or scissors 110 are magnetized, curvilinear needle 150 can be attached to needle manipulator 100 without using a tissue grasper to grasp the needle 150. Moreover, because an end of needle manipulator 100 can rotate, grasper 108 and scissors 110 will rotate. Thus, curvilinear needle 150 will also rotate, thereby enabling control of the orientation of curvilinear needle 150, keeping the tip of curvilinear needle 150 pointing in the same direction (for example, up or down).

For example, the end of needle manipulator 100 having grasper 108 and scissors 110 can be rotated along the Y-axis of curvilinear needle 150. This eliminate the need to rotate curvilinear needle 150 while grasping it with a tissue grasper and avoids having to rotate curvilinear needle 150 first on its Z-axis and then on its X-axis to change the directional orientation of curvilinear needle 150. A needle driver can then be used to take curvilinear needle 150, which is at the proper directional orientation, off of needle manipulator 100 and drive it through tissue.

In addition, because grasper 108 and/or scissors 110 is magnetized, they may be used to pick up curvilinear needle 150 or other metallic objects off of an organ, or from between the folds of an organ, without the need of a tissue grasper. This can prevent the inadvertent grasping and/or injury of an organ. Moreover, scissors 110 can be used to cut suturing material without having to remove needle manipulator 100 from the body and replace it with a cutting instrument, or create another access point for the cutting instrument. These advantages of needle manipulator 100 will improve the efficiency and safety of laparoscopic suturing and reduce the amount of time used to perform laparoscopic surgery.

Another embodiment of the needle manipulator includes adding a magnet to a conventional laparoscopic instrument, which would then have one or more of the following additional applications: rotating the needle in the same directional orientation, or magnetically picking up metallic objects such as needles, clips, or staples. In yet another embodiment, the needle manipulator is not magnetized and only has the grasper/scissor combination without the magnet.

Reference in the foregoing specification to “one embodiment” or “an embodiment” means that a particular feature, structure, or characteristic described in connection with the embodiment is included in at least one embodiment of the invention. The appearances of the phrase “in one embodiment” in various places in the specification are not necessarily all referring to the same embodiment.

In the foregoing specification, the invention has been described with reference to specific embodiments thereof. It will, however, be evident that various modifications and changes can be made thereto without departing from the broader spirit and scope of the invention. The specification and drawings are, accordingly, are to be regarded in an illustrative rather than a restrictive sense.

1. An apparatus, comprising:
a shaft;
a handle at a first end of the shaft; and
a grasper to hold an object at a second end of the shaft, wherein the handle controls opening and closing of the grasper.

2. The apparatus of claim 1, wherein the grasper includes scissors for cutting.

3. The apparatus of claim 1, wherein the shaft is rigid from the first end to the second end.

4. The apparatus of claim 1, further comprising an articulating joint to enable bending of an articulating portion of the shaft.

5. The apparatus of claim 4, wherein the articulating joint is located at a distance from an end of the shaft, and wherein a fixed portion of the shaft is coupled with a first part of the articulating joint and the articulating portion of the shaft is coupled with a second part of the articulating joint.

6. The apparatus of claim 5, wherein the handle comprises a wheel to cause the articulating portion of the shaft to bend.

7. The apparatus of claim 1, wherein the handle comprises a wheel to cause the grasper to rotate.

8. The apparatus of claim 1, wherein the handle is coupled with the shaft.

9. The apparatus of claim 1, wherein the handle is integrated as part of the shaft.

10. The apparatus of claim 1, wherein the grasper is integrated as part of the shaft.

11. The apparatus of claim 1, wherein the grasper is coupled with the shaft.

12. The apparatus of claim 1, wherein the grasper is magnetized to enable objects to attach to the grasper.
